(19) United States
(12) Patent Application Publication
(10) Pub. No.: US 2008/0109034 A1
(43) Pub. Date: May 8, 2008

(54) Controlled Adhesive Locations Facilitating Tissue Remodeling

(76) Inventors: Michael T. Mather, Mason, OH (US); Mark S. Ortiz, Milford, OH (US); Anil K. Nalagatla, Mason, OH (US); Frederick E. Shelton, Hillsboro, OH (US); James W. Voegele, Cincinnati, OH (US); Jeffrey S. Swayze, Hamilton, OH (US); Foster B. Stulen, Mason, OH (US); Binoy Bordoloi, Bridgewater, NJ (US); John V. Hunt, Cincinnati, OH (US)

Correspondence Address: FROST BROWN TODD, LLC, 2200 PNC CENTER, 201 E. FIFTH STREET, CINCINNATI, OH, 45202, US

(21) Appl. No.: 11/557,640

(22) Filed: Nov. 8, 2006

(51) Int. Cl. A61B 17/00

(52) U.S. Cl. 606/214

(57) ABSTRACT

A surgical implant for adhering two portions of tissue together with a polymer adhesive is disclosed. The surgical implant has a matrix structure with one or more layers and a plurality of holes for tissue growth therethrough. The matrix structure controls placement of the adhesive to minimize adhesive area and maximize tissue regrowth areas. In addition, the surgical implant can include drugs and adhesive initiators, and can include multiple layers of structure with any combination of holes, drugs, adhesives and adhesive initiators within. Additionally, the surgical implant can be surrounded by a rapidly dissolving pouch to prevent unwanted polymerization of the adhesive prior to placement in the body.

### FIELD OF THE INVENTION

The present invention relates, in general, to tissue fastening devices, and in particular to adhesives and adhesive systems for fastening tissue.

### BACKGROUND OF THE INVENTION

Tissue fastening can be either short term or long term duration. Short term duration fasteners can include a bandage, tape, removable staples, removable suture, adhesives, or absorbable stitches that are meant to provide temporary support until natural healing can occur.

Longer duration fasteners must remain in or on the body, possibly for the life of the patient. Longer duration fasteners include biocompatible implantables such as suture, staples, clips, tacks, clamps, pins, and the like. These long duration fasteners could be inserted subcutaneously in a surgical procedure and, after the patient has healed, cannot be removed without additional surgery. Longer term fasteners can provide short term and long term reinforcement for high force loads that can be 200-400% of normal forces. These high force loads could be caused by violent vomiting, coughing, and, in some cases, chronic overeating. For chronic overeaters that have undergone bariatric surgery to create a small stomach pouch, it is highly likely that a patient will “overload” the new pouch by attempting to eat the same large portions of food imbibed before the surgery.

Adhesives have been used topically as a short term fastener for wound repair. Closure Medical has developed a 2-octyl cyanoacrylate compound with a long carbon chain (eight carbons) that is biocompatible, has good bonding strength, and has received FDA approval for topical use. For short duration topical wound closure, the edges of the wound are brought together and at least one layer of the adhesive is applied along the surface of the wound line to form a barrier that holds the wound edges together. The cyanoacrylate adhesive also acts as a microbial barrier, keeping bacteria out and is eventually removed. Cyanoacrylate adhesives are described in United States Application 20040190975 by Goodman et al. which is herein incorporated by reference in its entirety.

Closure Medical is conducting an FDA clinical trial using a cyanoacrylate adhesive as an internal vascular tissue sealant and internal surgical adhesive. Some adhesives such as the cyanoacrylates, stick well to tissue, but like metallic fasteners, the fastener itself can become a local barrier to tissue regrowth through the fastener. For internal body use of surgical adhesives, the adhesive is used sparingly, not on top of the wound as in external use, but actually in the cut areas of the wound. By minimizing the glue areas across the wound, the surgeon is assured of maximum areas of tissue regrowth and minimal areas of the adhesive barrier. As the tissue regrows together and heals, the adhesive areas within the wound are encapsulated with healed tissue. Thus, internal adhesives are ideal for short term needs to hold cut tissue together so that healing can occur, and can remain as a long term fastener to provide additional strength to the healed tissue. Additionally, the adhesives can be biocompatable, bioabsorbable, and/or flexible, inside the body.

U.S. Pat. No. 6,004,333 by Sheffield Et al. discloses a prosthetic mesh with a plurality of collagen pads that can adhere the prosthetic mesh to tissue by an application of energy and pressure to the collagen pads. Energy and pressure must be applied to each and every collagen pad to create adhesion.

Consequently, a significant need exists for a long duration subcutaneous adhesive that can overcome the above-noted and other deficiencies of the prior art by providing significant attachment strength, minimize glue areas, minimize operative time, and promote tissue regrowth through the areas of adhesive application.

### BRIEF SUMMARY OF THE INVENTION

The invention overcomes the above-noted and other deficiencies of the prior art by providing a surgical implant for adhering two portions of tissue together. The surgical implant has at least one layer and a plurality of openings for tissue growth therethrough. A polymer adhesive is located about the implantable matrix for adhering the two portions of tissue together. The adhesive polymerizes to adhere the tissue together when the two portions of tissue are brought together.

Alternately a method of using a surgical implant to join two portions of tissue together is disclosed. The first step is providing an implantable matrix having a plurality of openings therein. The implantable matrix has at least one layer and an adhesive initiator. The second step is placing the implantable matrix onto a first portion of tissue. The third step is applying a polymer adhesive to the first portion of tissue and the implantable matrix. The fourth step is placing the second portion of tissue onto the first portion of tissue and the implantable matrix. And the last step is adhering the second portion of tissue onto the first portion of tissue and the implantable matrix by timed polymerization of the adhesive with the adhesive initiator.

And, yet another alternate method of using a surgical implant to join two portions of tissue together is disclosed. The first step of the method is providing an implantable matrix having at least one layer and a plurality of openings therein, and a polymerizable adhesive. The second step is placing the implantable matrix onto a first portion of tissue. The third step is placing the second portion of tissue onto the first portion of tissue and the implantable matrix. And the last step is adhering the second portion of tissue onto both the first portion of tissue and the implantable matrix by polymerizing the adhesive with tissue contact.

These and other objects and advantages of the present invention shall be made apparent from the accompanying drawings and the description thereof.

### BRIEF DESCRIPTION OF THE FIGURES

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 is an isometric view of a surgical implant with a plurality of dollops of adhesive or other compounds thereon.

FIG. 1a is a side view of FIG. 1.

FIG. 2 is an isometric view of sheet surgical implant with a plurality of dollops of adhesive or other compounds about the implant.

FIG. 2a is a side view of FIG. 2.

FIG. 3 is an isometric view of a portion of a suture implant with a plurality of stripes of adhesive or other compounds thereon.

FIG. 3a is a cross sectional view of the suture with adhesive stripes.

FIG. 4 is an isometric view of a plate structure implant in a pouch.

FIG. 5 is an isometric view of a mesh structure covered with adhesive and sealed in a pouch.

FIG. 6 is a cross sectional side view of a plurality of implantable structures assembled together with a plurality of adhesives and other compounds therein and sealed in a pouch.

FIG. 7 is an isometric view of a grasper placing a surgical implant having a plurality of dollops.

FIG. 8 is an isometric view of FIG. 7 with the grasper placing the surgical implant on a portion of tissue.

FIG. 9 is an isometric view of FIG. 8 with the grasper removed and an applicator placing a polymer adhesive over the surgical implant and tissue with the polymer adhesive being drawn by wicking to a structure of the surgical implant.

FIG. 10 is an isometric view of FIG. 8 with the applicator removed and the grasper placing a second portion of tissue over the surgical implant and adhesive.

FIG. 11 is an isometric view of two portions of tissue adhered together at a plurality of small points of adhesive and mesh with tissue growth to occurring between the adhesion points.

### DESCRIPTION OF THE INVENTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

During surgery, a variety of fasteners are typically used to approximate a wound by bringing two sides of tissue together in apposition to promote healing across the wound. Adhesives can be used as a surgical fastener, but like solid fasteners such as clips, staples, sutures and the like, the surgical fasteners themselves can be a barrier to tissue regrowth and healing. The physical size of the fasteners are a barrier to healing and could create an issue to longer term treatment. Minimizing the size of the fastener is one approach to this issue, and placing adhesives sparingly across the wound site embraces this approach. Minimal adhesive placement at a plurality of application sites can reduce the size of the adhesive barrier, but can require additional surgical time to carefully place the adhesive at a plurality of locations. By strategically pre-placing the adhesive, a single part of a two part adhesive, or an adhesive initiator on a surgically implantable structure that is biocompatible such as a hex matrix or a “web”, the adhesive barrier area is minimized, the healing area is maximized and the operative time to place the adhesive or initiator is minimized.

### Implant with Adhesives and Compounds

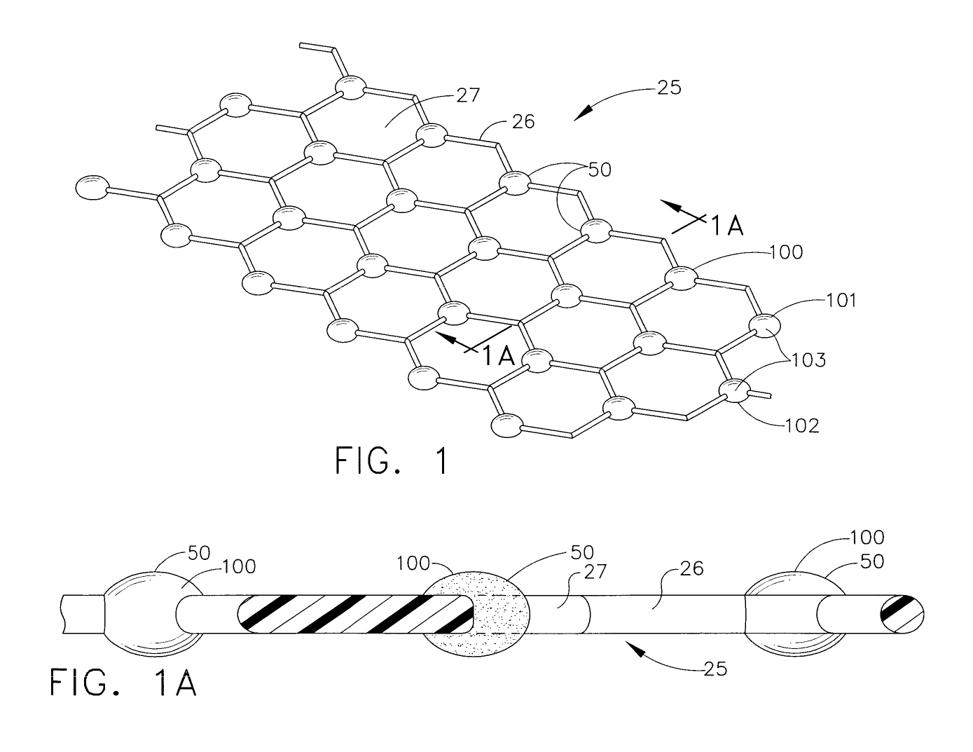

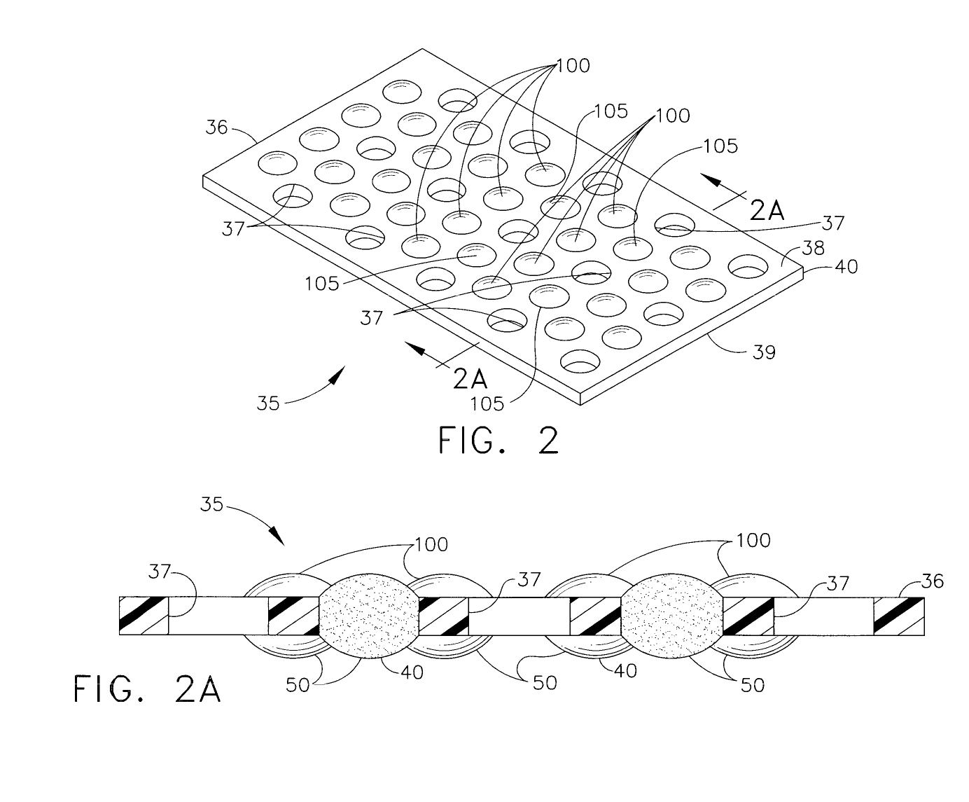

FIGS. 1 and 2 shows a surgical implant 25 comprising a structure 26, a plurality of openings 27 extending therethrough, and an adhesive 100 about said structure 26. Adhesive 100 is shown at a plurality of points or dollops 50 about the structure 26. Adhesive 100 can be a single part adhesive or can include a first part 101 or a second part 102 of a two part adhesive of tissue to surgical implant 25. Additionally, one or more of dollops 50 can include an adhesive initiator 104, or could have other alternate compounds 105 mixed with adhesives 100, 101, 102, 103. These alternate compounds and others will be described in greater detail below. Structure 26 with dollops 50 is shown rectangular but can be any shape such as circular, arcuate, triangular polygonal, and the like and can include one or more large openings therein, for example, to create a ring or a figure eight. Structure 26 can also be easily cut to fit any surgical site. Placement of structure 26 at the wound site rapidly places a number of spaced apart adhesion points (dollops 50) of adhesive 100, 101, 102, 103 that would adhere two portions of tissue together with minimal adhesives, yet provide a large area of tissue-to-tissue contact through the openings 27 to promote regrowth and tissue healing.

### Implantable Materials for a Structure

An implantable structure for the human body must be biocompatible, and can have many different properties. By way of example, an implatable structure such as structure 26 will now be described, and the material properties are not limited to structure 26 but can apply to any other structure or shape thereof. Structure 26 can be absorbable or non-absorbable. Absorbable materials 26a for structure 26 can include but are not limited to bioabsorbables such as polylactic acid, polyglycolic acid, polyglactin, polydioxanone, polyglyconate, whey protein, cellulose gum, starch, and gelatin. If the structure 26 is absorbable, structure 26 offers an additional benefit when the surgical goal is to maximize the healing area. The absorbable material used to form structure 26 would be naturally absorbed over time, and increase the area available for tissue regrowth and healing. Non-absorbable materials 26b suitable for implants such as structure 26 can include but are not limited to metallic materials such as stainless steel, titanium, and gold, and non-metallic materials such as silk, nylon, polypropylene, braided polyester, polybutester, polyethylene, and polyetheretherketones (PEEK).

Also by way of example, structure 26 can be any durometer or hardness to make structure 26 soft and pliable or firm to hard. For sites within a patient that undergo bending or require palpitation, the soft and pliable materials could be used. For sites within a patient that may require scaffolding, a firm or hard structure would be more appropriate. Additionally, the wall structure of structure 26 can be uniform or smooth, or cellular or textured. Smooth structures could have larger cross sections and higher strength than textured structures, but textured structures could allow cellular ingrowth into the textures. Additionally, by way of example, structure 26 could include image enhancing materials such as but not limited to barium or fluorescing additives. The imaging enhancing additives can increase or enhance visibility of the structure 26 to x-rays, ultrasound, light, and the like. Additional alternate compounds can be mixed with or used to coat structure 26 such as, but not limited to those described below as alternate compounds 105.

By way of example, structure 26 is shown as a thin flexible sheet structure with a plurality of holes. Alternately, sheet structure 26 can be any single thickness such as a mesh, plate, or a honeycomb or have more than one thickness. Additionally, by way of example, structure 26 can be any one of a number of shapes or structures such as a tube, a ball, an elongated “X”, a shape folded from a flat structure, a lattice, or any one of a number of shapes that can have at least one opening 27 and have adhesives attached thereto.

For a sheet structure such as structure 26 shown in FIGS. 1 and 2, the size and shape of the openings 27 can be any size or shape. For example, openings 27 can be, but are not limited to a hex, a circle, a triangle, and a rectangle. The size of openings 27 may be maximized to produce a minimal structure 26, and maximum area for tissue re-growth therethrough. Dollops 50 can be located about structure 26 at the intersections of the structure 26, at a plurality of points on the structure 26 for tissue contact, or at any point thereon including within openings 27. Dollops 50 can bulge above a structure, be flush with a structure, or concave into a structure. Whereas dollops 50 by way of example are shown as a circular or arcate shape, dollops 50 are not limited to that shape, can be any shape about the structure 26, and by way of example include a coating. Structure 26, can, for example, have wicking properties such that when placed on tissue, structure 26 can wick adhesive 100, 101, 102, 103 thereto. Wicking properties of structure 26 can ensure openings 27 remain clear of adhesive to allow tissue growth therethrough.

### Adhesives

Dollops 50 can be comprised of adhesives 100, 101, 102, 103, such as but not limited to polymerizable and/or cross-linkable materials such as a cyanoacrylate adhesive. The adhesive materials, for example, may be a monomeric (including prepolymeric) adhesive composition, a polymeric adhesive composition, or any other compound that can adhere to tissue. In embodiments, the monomer may be a 1,1-disubstituted ethylene monomer, e.g., an .alpha.-cyanoacrylate. When cross linked or polymerized, the cyanoacrylate can change from a liquid to a solid. Polymerized adhesives for example, can be formulated to be flexible to rigid. If desired, adhesives can be a single part or dual part adhesive, and/or can contain additives such as alternate compounds 105. Polymerization of the adhesives 100, 101, 102, 103 can occur from, but is not limited to, exposure to moisture or adhesion initiators 104.

### Alternate Compounds

Alternate compounds 105 can be attached on or about structure 26 as dollops 50, can be mixed with adhesives 100, 101, 102, 103, and could be attached to and/or combined with structure 26 in any other manner. Alternate compounds can include drugs, medicaments, contrasting agents, or any other commonly used surgical compounds, or any combination thereof. Examples of alternate compounds 105 can include, but are not limited to: adhesive initiators 104, image enhancing agents, necrosing agents, sclerosing agents, coagulants, theraputic agents, medicaments, analeptic agents, anesthesia agents, antidiuretic agents, analgesic agents, antiseptic agents, antispasmodic agents, cardiac agents, depressant agents, diuretic agents, hemostatic agents, hormonal agents, sedative agents, stimulant agents, vascular agents, time release agents, absorbable materials (see below, colorants, plasticizing agents, bulking agents, tamponade materials, thixotropic agents, antibacterial agents, buffers, catalysts, fillers, micro particles, thickeners, solvents, drugs, medicaments, natural or synthetic rubbers, stabilizers, pH modifiers, bioactive agents, cross-linking agents, chain transfer agents, fibrous reinforcements, colorants, preservatives, formaldehyde reducing or scavenging agents, flavorants, perfumes.

Adhesive initiators 104 are for polymerization and/or cross-linking of a polymerizable monomer. As used herein, a polymerization initiator is any material that causes a monomer composition applied to a substantially dry tissue (i.e., substantially in the absence of plasma or like tissue fluids) to polymerize in less than 300 seconds at ambient temperature, for example, at approximately 21-25.degree. C. Preferably, the initiator causes the monomer composition to polymerize in less than 150 seconds at ambient temperature, more preferably within 60, 90 or 130 seconds. As used herein, a polymerization rate modifier is any material that changes the rate at which a polymerizable monomer would polymerize in the absence of that material. Preferably, the rate modifier accelerates the rate of the polymerization reaction, although for particularly fast-acting monomers it may decelerate that rate.

Particular initiators 104 for particular monomers may be readily selected by one of skill in the art without undue experimentation. Control of the molecular weight distribution of the applied adhesive can be enhanced by selection of the concentration and functionality of the initiator or accelerator vis-a-vis the selected monomer. Suitable polymerization initiators and accelerators for cyanoacrylate compositions include, but are not limited to, detergent compositions; surfactants, including nonionic surfactants such as polysorbate 20 (e.g., Tween 20.™.; ICI Americas), polysorbate 80 (e.g., Tween 80.™.; ICI Americas), and poloxamers; cationic surlactants such as tetrabutylammonium bromide; anionic surfactants, including quaternary ammonium halides such as benzalkonium chloride or its pure components, and benzethonium chloride; stannous octoate (tin (II) 2-ethylhexanoate), and sodium tetradecyl sulfate; and amphoteric or zwitterionic surfactants such as dodecyldimethyl(3-sulfopropyl) ammonium hydroxide, inner salt; amines, imines, and amides, such as imidazole, tryptamine, urea, arginine and povidine; phosphines, phosphites and phosphonium salts, such as triphenylphosphine and triethyl phosphite; alcohols such as ethylene glycol; methyl gallate; inorganic bases and salts, such as sodium bisulfite, magnesium hydroxide, calcium sulfate and sodium silicate; sulfur compounds such as thiourea and polysulfides; polymeric cyclic ethers such as monensin, nonactin, crown ethers, calixarenes and polymeric epoxides; cyclic and acyclic carbonates, such as diethyl carbonate; phase transfer catalysts such as Aliquat.™. 336 (General Mills, Inc., Minneapolis, Minn.); organometallics; manganese acetylacetonate; radical initiators and radicals, such as di-t-butyl peroxide and azobisisobutyronitrile; and bioactive compounds or agents.

Alternately, the initiator may be a bioactive material, including quaternary ammonium halides such as alkylbenzyldimethylammonium chloride (benzalkonium chloride; BAC) its pure components, or mixtures thereof, especially those with an alkyl containing 6-18 carbon atoms; benzethonium chloride; and salts of sulfadiazine. Cobalt napthenate can be used as an accelerator for peroxide. Other suitable bioactive materials are disclosed in U.S. Pat. No. 5,928,611 to Leung and U.S. patent application Ser. No. 08/920,876, filed Aug. 29, 1997, Ser. No. 09/430,176 filed Oct. 29, 1999, and Ser. No. 09/430,177, filed Oct. 29, 1999, the entire disclosures of which is incorporated herein by reference.

Other examples of adhesives 100, 101, 102, 103, adhesive initiators 104, and alternate compounds 105 may be found in United States Application 20040190975 by Goodman et al. which is herein incorporated by reference in its entirety.

### Honeycomb Structure

One embodiment of an alternate surgical implant 35 can have an absorbable plate structure or honeycomb 36 having a top surface 38, a bottom surface 39, and including a plurality of openings 37 extending therethrough. Honeycomb 36 can be any height 40 and can contain scorings or other modifications such as stress risers to induce breakup and absorption. Openings 37 can be empty for tissue growth, contain adhesives 100, 101, 102, 103, contain adhesive initiators 104, and contain alternate compounds 105, in any combination or location.

For example, as shown in FIG. 2, surgical implant 35 adhesive 100 is placed into every other row. In alternate rows, empty openings 37 are provided for tissue regrowth, and alternate openings 37 in non-adhesive rows are shown filled with an alternate compound 105 such as a time release drug or other substance, and if desired, can be mixed with an adhesive, neat, or compounded with other materials. As shown in cross sectional view of FIG. 2a, the rearmost row of openings 37 contain adhesive 100, and the foremost row of openings 37 alternate empty openings 37 with alternate compounds 105.

Additionally, by way of example, adhesive 100 could be applied to each surface 38, 39 of honeycomb 36, and some or all of openings 37 could be left open to provide tissue healing areas. Thus, any combination of a structure 36 with openings 37, adhesives 100, a first part 101 or a second part 102 of a two part adhesive 103, adhesive initiators 104 or an alternate compound 105 described above could be used, and the above examples are not meant to be limiting in any way. Honeycomb 36 can be easily fabricated by stamping holes in a sheet, molding, extrusion, laser or abrasive cutting, or any one of a number of commercial processes, and can be any shape described above.

Surgical implant 35 could work differently in the patient than the above surgical implant 25. For example, if honeycomb 36 of surgical implant 35 is constructed from an absorbable material listed above, and some or all openings 37 are filled with dollops 50 of adhesive 100, placement of surgical implant 35 between two layers of tissue would adhere tissue to adhesive 100. The absorbable honeycomb 36 itself could create a temporary barrier within the body to healing and regrowth, but as the absorbable material of honeycomb 36 is absorbed, the barrier to tissue growth is removed, and only dollops 50 of adhesive 100 would remain from surgical implant 35 holding the tissue together. If the dollops 50 of adhesive 100 are bioabsorbable, then full tissue healing can occur. If desired, barbs or attachment features could be included attached to honeycomb 36 and could be made from similar or dissimilar materials. Alternately, mechanical or suture ‘tacking’ could be used as an alternative to barbs.

Alternately, by way of another example, honeycomb could have many other uses other than as a tissue adhesion device. If some openings 37 of honeycomb 36 could dollops 50 of adhesives 100, 101, 102, 103, the additional openings 37 could contain dollops 50 of alternate compound such as a time release drug. The drug would be eluted slowly from the honeycomb 36 and/or, as honeycomb 36 structure dissolves. Thus, alternate surgical implant 35 combined with adhesive 100 could be used to adhere tissue, act as a drug reservoir to medicate tissue, used as a bandage, a sealing means, an attachment device for other components or devices, or any combination thereof.

For the honeycomb 36, different thickness, different absorbable materials or differing fractions of their concentration can be used to vary the time at which the contents are released. Incorporated materials or the walls themselves could dissolve at different rates associated with pH (base or acid). This could have some gastric applications.

A similar approach may be to incorporate PEGs, PLAs or dendrimers or similar materials with ligands into the walls of the honeycomb 36. When appropriate molecules or cells (cancer) come in contact with the ligands, a morphological change or dissolution of the larger macromolecule would occur to release the contents.

Releasing drugs or medicaments on demand is desirable since it could be based on the individual patient's response to a given treatment. An appealing approach is to use an excorporeal energy source to rupture or otherwise disrupt the integrity of the cells or openings 37 of honeycomb 36. It is envisioned that these would require the incorporation of special materials that are receptive to the particular form of energy. Energy could also be delivered if appropriate through natural orifices. Sources can include: laser, electromagnetic (susceptibility), and/or ultrasound.

Laser sources would require that an appropriate receptor dye be in the honeycomb material that would be highly absorptive of the energy at the laser's wavelength. Very precise control of the laser would allow very small dense packing of honeycomb cells. It may be possible to first “site” the desired cell with one wavelength or “amplitude” and then repute the wall with more energy or a different wavelength. This approach would be most appropriate were “line of sight” is available such as natural orifices and dermal applications where the honeycomb is buried under the skin such as treatment of the prostate.

Magnetic susceptibility materials have been suggested for use in oncology. Once the material is forced into a tumor, it is heated by applying an external electromagnetic field. The material could heats to ablate/kill the tumor. The same material could be incorporated in the construction of the honeycomb walls. One could image that for the cells to be ruptured early in the treatment the wall would be doped with more of the material. So they would burst first. For cells to be ruptured later in the treatment regiment, their cells would be doped with less material.

Ultrasound can be a very viable option for selective and on-demand release of drugs and medicaments from honeycomb 36. Ultrasound can be focused to a very small volume, which is the basis for diagnostic medical ultrasound. Therefore it can be very selective in terms of targeting specific cells in the honeycomb 36. The dimension of the cell could be slightly greater than the focus spot size of the ultrasound system.

One can image that an ultrasonic diagnostic unit first images the honeycomb to locate the desired cell of honeycomb 36. It is then switched to focus on the wall. The power is increased to bring the amplitude level well above the cavitation threshold. Therefore surrounding interstitial fluids will cavitate and implode near the surface of the desired cell to disrupt the cell.

Cavitation damage can be enhanced by first using a more intensity and higher frequencies to create a cavitation swarm near the surface, and then apply a lower frequency and/or lower power to sustain the cavitation for a longer period of time. These frequencies and or amplitudes are cycled at an appropriate pulse(s) repeat cycle to maximize the damage.

To enhance this approach microspheres containing fluids or gases can be incorporated into the wall material of the honeycomb 36. This has two actions. At lower diagnostic amplitudes it well make the honeycomb 36 structure easier to see. At higher amplitudes the microspheres will burst and thereby rupture the wall.

Another ultrasonic approach is to incorporate a highly ultrasonic absorptive material into the honeycomb 36. In this case the ultrasonic is used to heat the honeycomb 36 to soften and melt it. The potential temperature rise would need to be compatible with the contents of the cell.

Rectified diffusion is an ultrasonic mechanism that grows gas bubbles from gases normally held in solution. This mechanism is incorporated by introducing a dissolved gas such as CO2 to the contents (liquid) or into the wall (depending on the native strength of the wall). Therefore either the wall or the contents would expand under the influence of ultrasound and thereby rupture the cell of the honeycomb 36. In this approach the volume of cell needs to be greater than the focus volume so that one cell at at time could be burst.

The wall or the volume of the cell can be designed to be resonant at a given frequency. When that cell is impacted by an ultrasound wave of that frequency, it begins to resonant as the vibrational amplitude increases, the internal stresses increases so that cell ruptures.

An interesting concept is to first disrupt thinner adjoining walls of honeycomb 36 cells of agents that need to be mixed to be effective when released. This approach could potentially first rupture the inner wall and mix the contents, then higher intensity would be used to rupture the outer walls.

Microstreaming and shear-thinning are two ultrasonic mechanisms. These could be used to speed the dissolution of a bioabsorbable. The ultrasound would be focused on the targeted cell of the honeycomb 36 for a period of time. By these mechanisms the rate of degradation would significantly increase.

Any combination of some or all of these mechanisms are possible and may allow for a convenient system for en masse release or targeted release. Likewise other energy modalities may also provide means to release and deliver the therapeutics contained within the honeycomb 36.

### Suture Mesh Structure

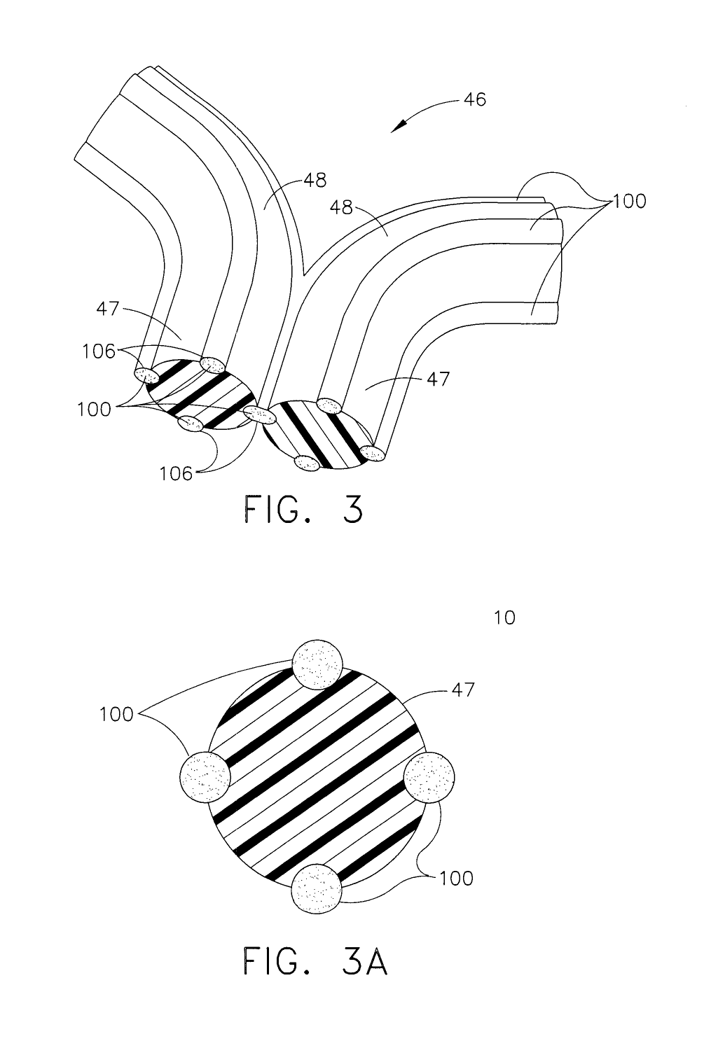

FIGS. 3 and 3a shows a small portion of an alternate embodiment of a wavy mesh structure 46 constructed from a suture 47. Suture is commonly used by surgeons as an implantable fastener and may be absorbable or non-absorbable. Suture 47 can be manufactured by an extrusion process through a spinnerette to align the long chain molecules and create a maximum strength structure. As shown in FIG. 3, stripes 106 of adhesive 100 could also be co-extruded onto suture 47 or applied in a secondary process The suture 47 with adhesive stripes 106 can be bent, formed, or woven into sinusoidal lengths 48 that can be adhered together to form a mesh structure by assembling adjacent sinusoidal lengths 48 together. Where the adhesives 100 touch, the suture 47 is joined. In FIG. 3, the suture 47 and stripes 106 of the two sinusoidal lengths 48 shown are pressed together allowing the two stripes 106 to join. These wavy lengths could form a sinusoidal mesh structure 46 or, if the sinusoidal lengths 48 is formed differently, a hex mesh structure can be formed similar to that shown in FIG. 1. When the wavy mesh structure 46 is used, large areas of open tissue-to-tissue contact are available for healing, while tissue portions brought into contact with the adhesive stripes 101 on suture 46 are bonded thereto. Suture 101 could be absorbable or non-absorbable. Absorbable suture 47 would not only allow for tissue regrowth around the suture 47 and adhesive stripes 101 in the bonded areas, but through the bonded areas as well, if the adhesive stripe 101 was designed to degrade and be absorbed by the body.

Whereas the suture 47 is shown with all stripes 106 formed from adhesive 100, each of the stripes 101 could be a single material, or a different material or ay combination thereof. Thus, stripes 106 and can include adhesives 100, a first part 101 or a second part 102 of a two part adhesive 103, adhesive initiators 104, or alternate compounds 105. Stripes 106 can be formed neat from any of the compounds 100, 101, 102, 103, 104, 105 or combined in any proportion with plastics such as any biocompatible plastic mentioned above.

### Structure with Adhesives and Pouch

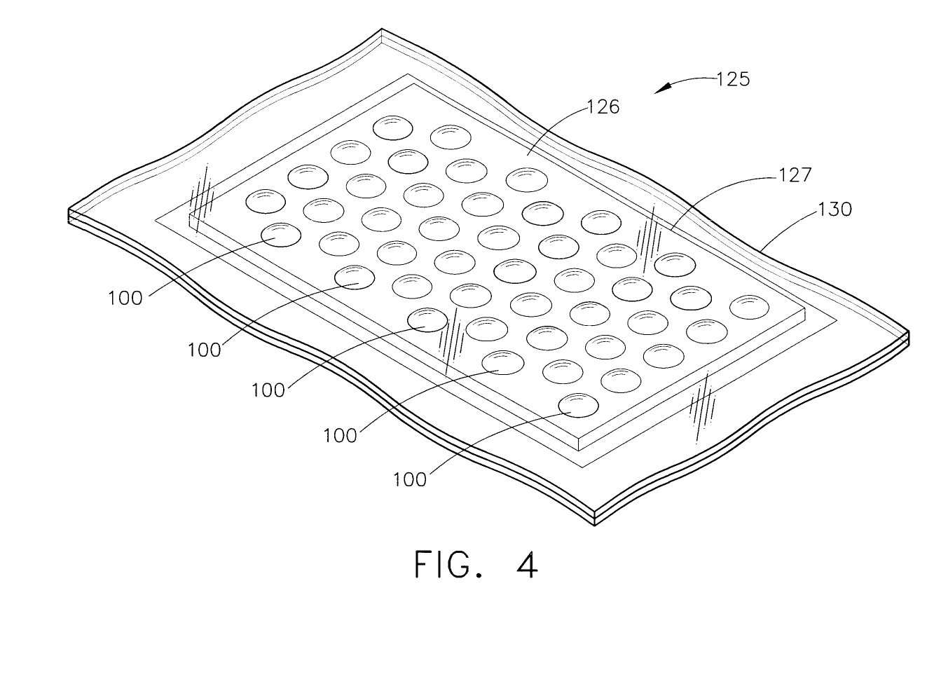

FIG. 4 shows an alternate embodiment of a surgical implant 145 comprising a pouch 130 containing a structure 126 having openings 127 and containing dollops 50 of any combination of compounds 100, 101, 102, 103, 104 and 105 therein. Pouch 130 is sealed and acts as a barrier to prevent oxygen, moisture, ozone, dirt, dust, hair, contaminants, bacteria, viruses and the like from contaminating or degrading the structure 126 or any compounds 100, 101, 102, 103, 104 and 105. Thus, pouch 130 can prevent contamination of the contents, increase the shelf life of the contents, and can constrain or prevent oozing and migration of the contents.

As shown, pouch 130 is made from an absorbable material and can be planted directly into a wound site such as polylactic acid, polyglycolic acid, polyglactin, polydioxanone, and polyglyconate. Additionally, pouch 130 could be made from an absorbable material that is a meltable or rapidly dissolving film such as those used for breath strips. Absorbable materials that could melt or dissolve or liquefy can include materials such as but not limited to whey protein, cellulose gums, starches, gelatins, carboxymethyl cellulose (CMC), polyethylene glycol (PEG), hyaluronic acid (HA), or other compounds. As shown, pouch 130 is a rapidly dissolving film, can be placed onto tissue, and could be dissolved by body moisture, by the application of saline, by the application of an adhesive initiator, or by the application of any one of a number of other chemicals or compounds. If desired, pouch 130 could be unsealed, sealed, or could comprise a pair of sheets 131 (not shown) that adhere to the structure 126 or dollops 50. Sheets 131 could be absorbable for implanting, or if non-absorbable, removed prior to placement into the body.

Alternately, pouch 130 could be made from one or more layers. Multi-layer pouches by way of example could be made from multiple layers with each layer performing a desired function. For example, one layer of pouch 130 could be a moisture barrier of low strength attached to a porous second layer of high strength. The combination of layers could provide a strong pouch 130 that can prevent moisture passage therethrough

Whereas the above pouch 130 is taught as being combined with structure 126, its usage is not limited to just structure 126 but can include any shape of structure described above such as structure 26, structure 36, or structure 46, and any combination of dollops 50 of adhesives 100, a first part 101 or a second part 102 of a two part adhesive 103, adhesive initiators 104 and any alternate compound 105 used therein. Additionally, any structure used in pouch 130 could be absorbable materials 26a or non-absorbable materials 26b.

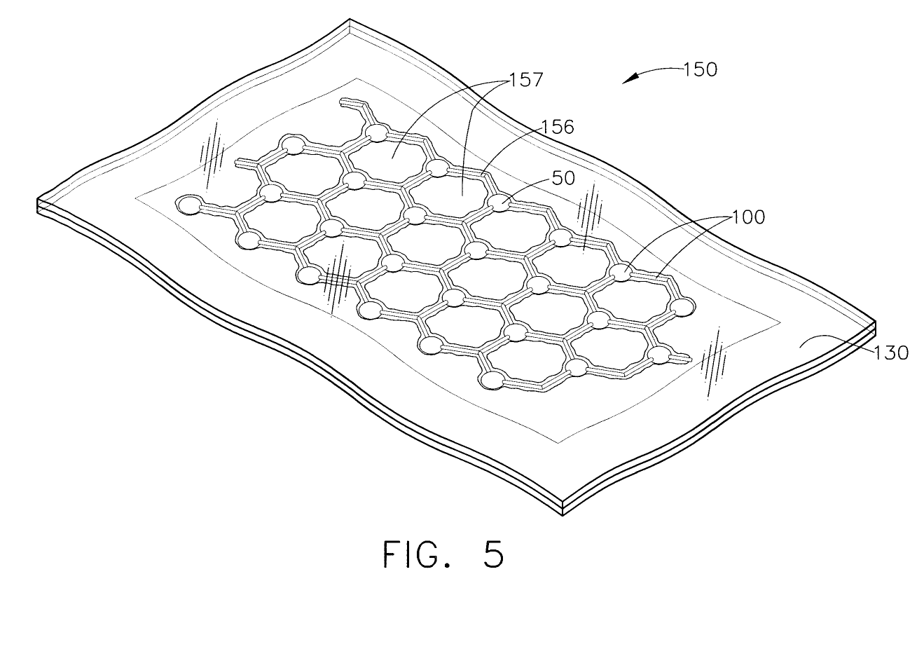

FIG. 5 shows one example of a surgical implant 150 comprising a non-absorbable pouch 150 containing a structure 156 having openings 157 and dollops 50 with a coating of adhesive 100 wicked about the structure 156. Pouch 150 can be constructed from non-absorbable materials such as but not limited to polyethelene, polypropelene or any one of a number of compatible materials. Pouch 150 exemplarily keeps adhesive 100 fresh and prevents contamination. Pouch 150, if non-absorbable, must be removed and discarded prior to placement of structure 156 into the patient and pouch 150 can be single or multiple layers. Dollops 50 could be any compounds 100, 101, 102, 103 or 105. As shown, both the structure 156 and the adhesive 100 are non-absorbable.

### Multi-Layer Structures with Compounds

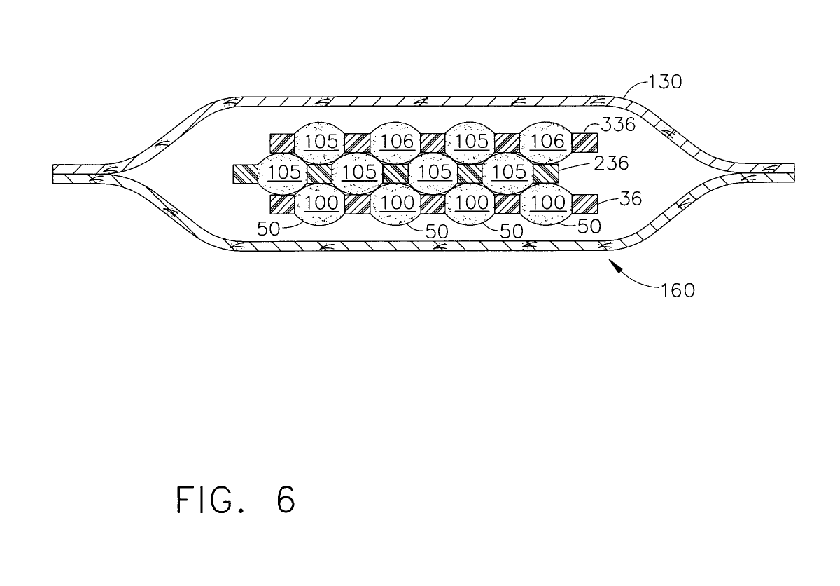

And, as shown in the side view of FIG. 6, a surgical implant 165 could be constructed from two or more combinations of layers of structures. As shown surgical implant 165 is formed from three layers of absorbable structures such as structure 36, structure 236, and structure 336 to form an assembled structure 166. Each layer of structure 166 has one or more openings 27 therethrough, and some combination of empty openings 27 and dollops 50 of any combination of compounds 100, 101, 102, 103, 104, and 105 therein. Each layer of structure 166 could be placed on top of another layer in any orientation to create a layered sandwich type construction with uniform openings passing through the layers, or as shown in FIG. 6, the layers could be staggered to create labyrinth openings. Compounds 100, 101, 102, 103, are used to adhere to tissue and/or to hold structure 166 together, and compounds 104, 105 can elute from structure 166 and/or hold layers together. Thus surgical implant 165 can be a structure comprising a number of assembled layers that slowly exposes compounds 105 as the structure 166 is absorbed. Labyrinth openings could slow the elution of compounds 105 therefrom. Additionally, flat plates 168 (not shown) of an absorbable structure material could be used during the construction of surgical implant 166.

As shown in FIG. 6, surgical implant 160 is constructed from structures 36, 236, and 336 and placed in a pouch 130 with layer 236 staggered relative to layers 36 and 336. Dollops 50 are placed in the structures 36, 236, and 336, and include a layer of dollops 50 of adhesive 100 in structure 36, a layer of dollops 50 of an alternate compound 105 in structure 236 and in structure 336, alternating dollops 50 of a first alternate compound 105 and a second alternate compound 106. First alternate compound 105 and a second alternate compound 106 are mixed with an adhesive 100. Placement of the layers together enables dollops of adhesive 100, first alternate compound 105, and second alternate compound 106 to adhere the surgical implant together and, where exposed, to tissue. Alternately, dollops 50 may be aligned be aligned or overlap or isolated to create unique chambers. Such a surgical implant 160 could have a variety of uses including wound repair, and/or as a time release dispersant of drugs.

### Method of Use of a Surgical Implant

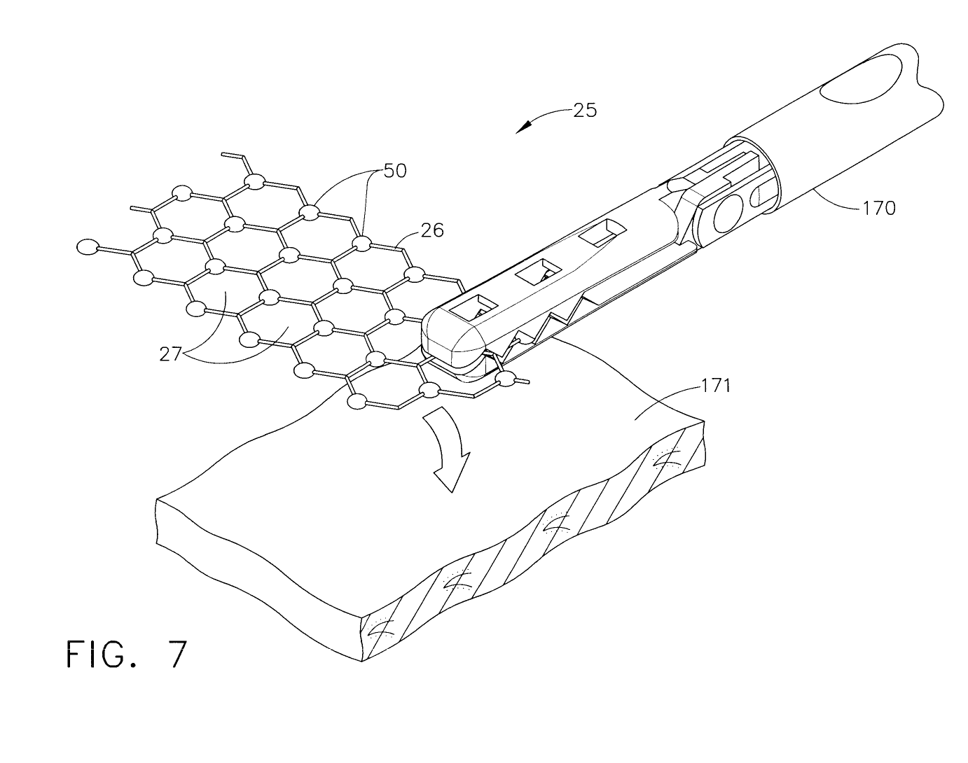

FIGS. 7-12 show an example of a surgical implant 25 being used to adhere two portions of tissue together. FIG. 7 shows a grasper 170 bringing a surgical implant 28 to a wound site with a first tissue layer 171. Surgical implant comprises a structure 26, a plurality of openings 27 extending therethrough, and a plurality of dollops 50 attached at corners of the structure. Dollops 50 for this example are adhesive initiators 104 attached to structure 26.

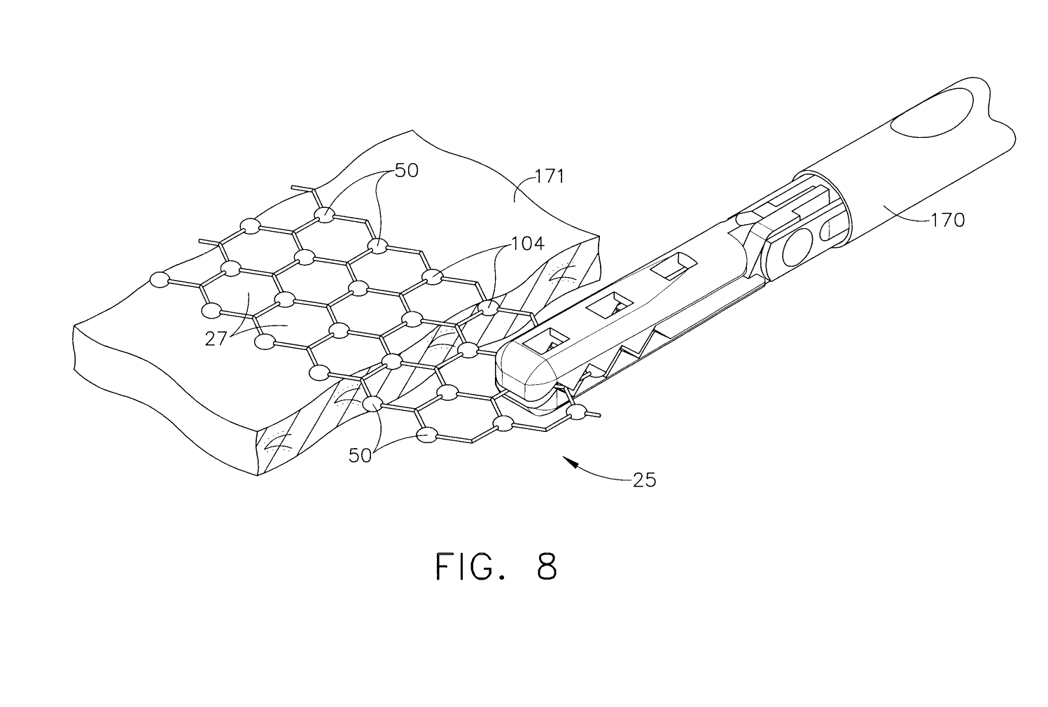

FIG. 8 shows a surgeon positioning the placement of surgical implant 25 onto tissue with a grasper 170 just prior to release the of the surgical implant 25. Dollops 50 and structure 26 of surgical implant are in contact with tissue.

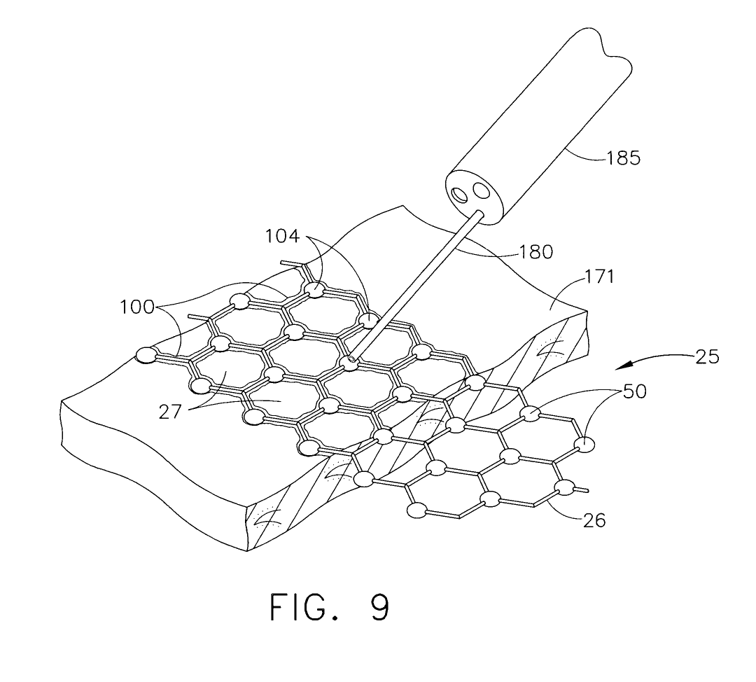

FIG. 9 shows the placement of an adhesive 100 onto the surgical implant 25. Adhesive 100 is being dispersed from a needle 180 extending from a working channel of an endoscope 185. Adhesive 100, by way of example, is a thin non-viscous cyanoacrylate that when applied over surgical implant 25 and tissue layer 171 flows freely over tissue 171 and is attracted to the implant 126 by capillary or wicking action. This wicking action pulls adhesive 100 off of tissue 171 about openings 27 and to implant 25 as shown.

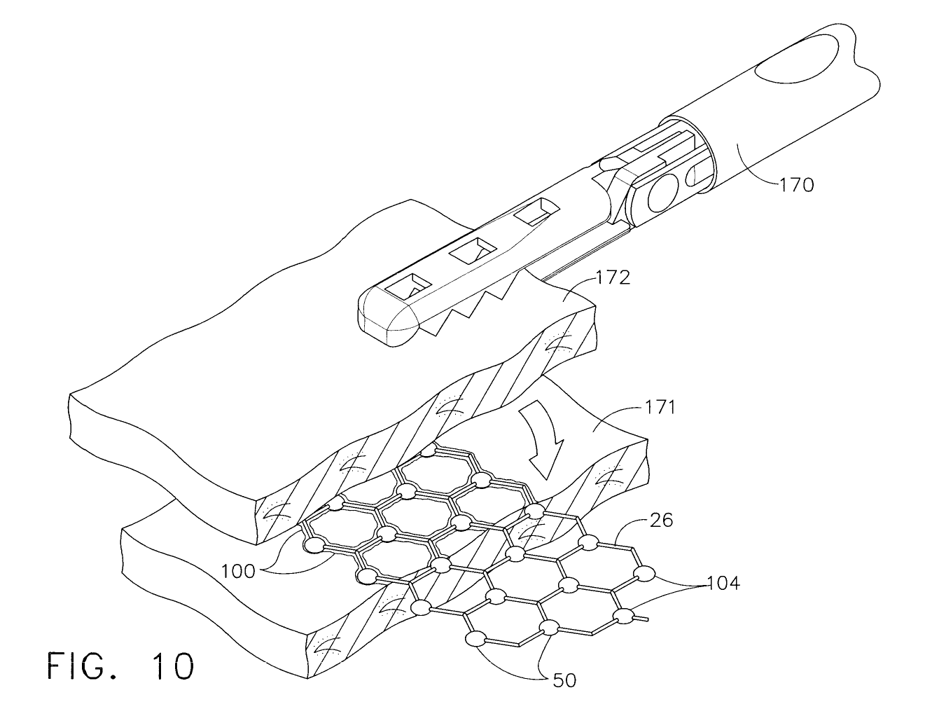

A time, depending on the compounding of adhesive initiator 104, is available to the surgeon to place a second layer of tissue 172 over the first layer of tissue 171, surgical implant 26, and adhesive 100 before adhesive initiators 104 begin to set the adhesive 100. FIG. 10 shows a second portion of tissue 172 being placed over surgical implant 25 and adhesive 100 prior to polymerization of adhesive 100.

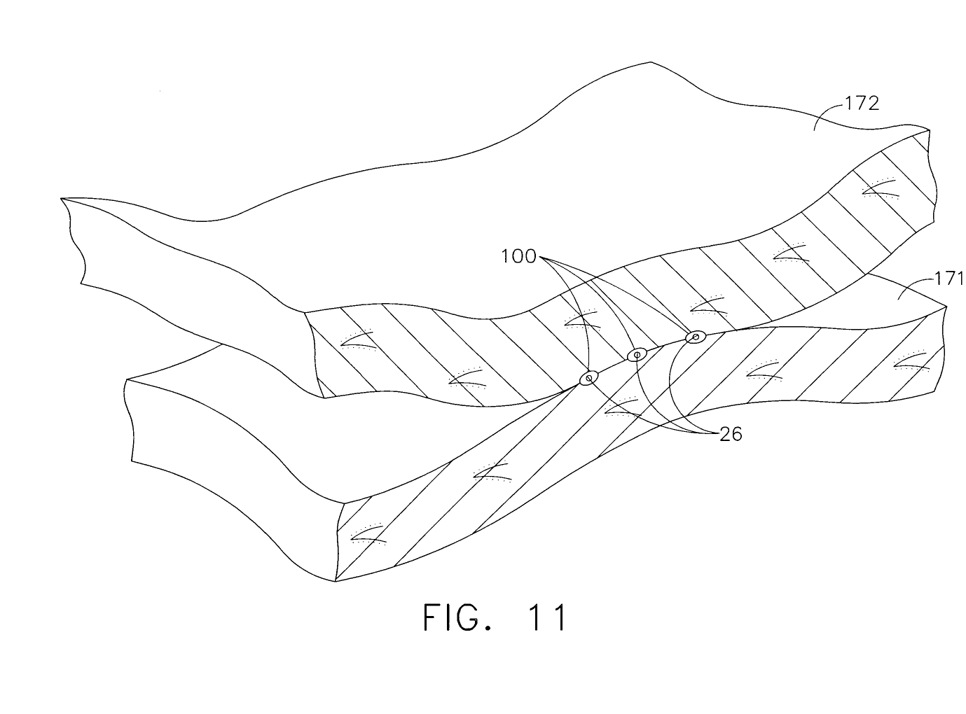

FIG. 11 shows a cross section of the tissue 171, 172 after being adhered together by adhesive 100. Three small cross sectional areas of set up cyanoacrylate adhesive 100 and structure 26 are shown holding tissue 171, 172 together. Large areas of tissue contact 173 are available for tissue healing and regrowth.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

While the present invention has been illustrated by description of several embodiments and while the illustrative embodiments have been described in considerable detail, it is not the intention of the applicant to restrict or in any way limit the scope of the appended claims to such detail. Additional advantages and modifications may readily appear to those skilled in the art.

For example, whereas several combinations of elements and examples are cited above, it would obvious to one skilled in the art to make other combinations from the elements listed above that would fall into the spirit of the present invention such as but not limited to: an absorbable ball structure containing drugs released by laser energy and sealed within a pouch.

What is claimed is:

1. A surgical implant for adhering two portions of tissue together comprising;
a) an implantable matrix having at least one layer and a plurality of openings formed within the at least one layer for tissue growth therethrough; and
b) a polymer adhesive about the implantable matrix for adhering the two portions of tissue together, the adhesive polymerizing to adhere the tissue together when the two portions of tissue are brought together.

2. The surgical implant of claim 1 wherein the polymer adhesive is polymerized by a selected one of a group consisting of moisture and an adhesive initiator.

3. The surgical implant of claim 2 wherein the polymer adhesive is at least one selected from a group consisting of polymerizable monomer, a polymerizable 1,1,1,1-disubstituted ethylene monomer, and a cyanoacrylate formulation.

4. The surgical implant of claim 3 further comprising a compound selected from a group consisting of a plasticizing agents, a thixotropic agent, an antibacterial agent, a buffer, a catalyst, filler, a micro particle, a thickener, solvent, a marking agent, an image enhancing agent, am theraputic drug, a time release agent, a tamponade material, and a bulking agent.

5. The surgical implant of claim 4 wherein the selected compound is located about the implantable matrix at a location selected from a group consisting of mixed into the surgical matrix, at a plurality of spaced apart points about the surgical matrix, within at least one of the plurality of openings, and as a coating surrounding the implantable matrix.

6. The surgical implant of claim 4 wherein at least one of the adhesive and the implantable matrix and the alternate compounds are absorbable.

7. The surgical implant of claim 4 wherein at least one layer of the implantable matrix includes a floor blocking at least one of the plurality of openings.

8. The surgical implant of claim 4 wherein the surgical implant is sealed in a pouch dissolvable on contact with tissue and moisture, the pouch constructed from at least one material selected from a group consisting of polylactic acid, polyglycolic acid, whey protein, cellulose gums, starches, gelatins, carboxymethyl cellulose, polyethylene glycol, and hyaluronic acid.

9. The surgical implant of claim 1 wherein the implantable structure is constructed from at least one material selected from a group consisting of polyethylene polymers, polyetheretherketones, polylactic acid, polyglycolic acid, polyglactin, polydioxanone, polyglyconate, whey protein, cellulose gum, starch, gelatin, carboxymethyl cellulose, polyethylene glycol, and hyaluronic acid.

10. A method of using a surgical implant to join two portions of tissue together comprising;
a) providing an implantable matrix having a plurality of openings therein formed within the at least one layer for tissue growth therethrough, the implantable matrix having at least one layer and an adhesive initiator;
b) placing the implantable matrix onto a first portion of tissue;
c) applying a polymer adhesive to the first portion of tissue and the implantable matrix;
d) placing the second portion of tissue onto the first portion of tissue and the implantable matrix; and
e) adhering the second portion of tissue onto the first portion of tissue and the implantable matrix by timed polymerization of the adhesive with the adhesive initiator.

11. The method of claim 10 wherein the polymer adhesive is polymerized by least one selected from a group of a polymerizable monomer, a polymerizable 1,1,1,1-disubstituted ethylene monomer, and a cyanoacrylate formulation.

12. The method of claim 10 wherein the implantable matrix has at least one wicking feature and the step of applying the adhesive includes wicking the adhesive to the implantable matrix to clear the plurality of openings of adhesive.

13. The method of claim 10 further comprising the step of regrowing tissue through the plurality of openings.

14. The method of claim 10 wherein the implantable matrix further comprises at least one alternate compound selected from a group of a drug, an agent, and a medicament and including a step of dispensing the alternate compound to tissue over time.

15. The method of claim 10 wherein the implantable matrix further comprises at least one imaging compound and further including a step of locating the surgical site with an imaging apparatus after tissue regrowth.

16. The method of claim 10 wherein one or more of the adhesive and the implantable matrix is absorbable and further including a step of absorbing the absorbable one or more of the adhesive and the implantable matrix to allow additional tissue regrowth.

17. The method of claim 10 wherein the implantable matrix has at least one barb and the step of placing the implantable matrix and the step of placing the second portion of tissue includes a step of placing the at least one barb into tissue.

18. A method of using a surgical implant to join two portions of tissue together comprising;
a) providing an implantable matrix having at least one layer and a plurality of openings formed within the at least one layer for tissue growth therethrough and a polymerizable adhesive;
b) placing the implantable matrix onto a first portion of tissue;
c) placing the second portion of tissue onto the first portion of tissue and the implantable matrix; and
d) adhering the second portion of tissue onto the first portion of tissue and the implantable matrix by polymerizing the adhesive with tissue contact.

19. The method of claim 18 wherein the adhesive is at least one selected from a group consisting of a polymerizable monomer, a polymerizable 1,1,1,1-disubstituted ethylene monomer, and a cyanoacrylate formulation.

20. The method of claim 18 wherein the implantable matrix further comprises at least one alternate compound selected from a group consisting of a drug, an agent, and a medicament and including a step of dispensing the alternate compound to tissue over time.

21. The method of claim 18 wherein one or more of the adhesive and the implantable matrix is absorbable by tissue and further including a step of absorbing the absorbable one or more of the adhesive and the implantable matrix to allow additional tissue regrowth through the absorbed matrix.

22. The method of claim 18 further including a pouch surrounding the implantable matrix wherein the step of placing the implantable matrix onto a first portion of tissue includes the step of dissolving the pouch with one or more selected from the group consisting of tissue contact and moisture to expose the implantable matrix and polymerizable adhesive to tissue contact.
